(19) United States
(12) Patent Application Publication
(10) Pub. No.: US 2003/0220526 A1
(43) Pub. Date: Nov. 27, 2003

(54) Iodination of 4-fluoro-benzaldehyde

(76) Inventors: Gregory S. Wayne, Vernon Hills, IL (US); Steven J. Wittenberger, Mundelein, IL (US); Sou-Jen Chang, Prairie View, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/368,798

(22) Filed: Feb. 13, 2003

Related U.S. Application Data
(60) Provisional application No. 60/357,892, filed on Feb. 19, 2002.

(51) Int. Cl. C07C 45/63

(52) U.S. Cl. 568/426

(57) ABSTRACT

The present invention relates to an improved process for iodinating a substituted benzaldehyde.

This application claims priority to the U.S. provisional application Ser. No. 60/357,892, filed Feb. 19, 2002.

### BACKGROUND OF THE INVENTION

Potassium channels play an important role in regulating cell membrane excitability. When the potassium channels open, changes in the electrical potential across the cell membrane occur and result in a more polarized state. A number of diseases or conditions may be treated with therapeutic agents that open potassium channels; see for example (K. Lawson, Pharmacol. Ther., v. 70, pp. 39-63 (1996)); (D. R. Gehlert et al., Prog. Neuro-Psychopharmacol & Biol. Psychiat., v. 18, pp. 1093-1102 (1994)); (M. Gopalakrishnan et al., Drug Development Research, v. 28, pp. 95-127 (1993)); (J. E. Freedman et al., The Neuroscientist, v. 2, pp. 145152 (1996)); (D. E. Nurse et al., Br. J. Urol., v. 68 pp. 27-31 (1991)); (B. B. Howe et al., J. Pharmacol. Exp. Ther., v. 274 pp. 884-890 (1995)); (D. Spanswick et al., Nature, v. 390 pp. 521-25 (Dec. 4, 1997)); (Dompeling Vasa. Supplementum (1992) 3434); (WO9932495); (Grover, J Mol Cell Cardiol. (2000) 32, 677); and (Buchheit, Pulmonary Pharmacology & Therapeutics (1999) 12, 103). Such diseases or conditions include asthma, epilepsy, male sexual dysfunction, female sexual dysfunction, pain, bladder overactivity, stroke, diseases associated with decreased skeletal blood flow such as Raynaud's phenomenon and intermittent claudication, eating disorders, functional bowel disorders, neurodegeneration, benign prostatic hyperplasia (BPH), dysmenorrhea, premature labor, alopecia, cardioprotection, coronary artery disease, angina, ischemia, and incontinence.

4-fluoro-3-iodo-benzaldehyde was previously synthesized in five steps as shown in Scheme 1. In summary, 4-fluorobenzoic acid (1) is converted to 4-fluoro-3-nitrobenzoic acid (2) which is then reduced to the corresponding amine (3). Further reduction results in the alcohol (4) and subsequent iodination (5) and oxidation ultimately leads to the iodinated benzaldehyde (6).

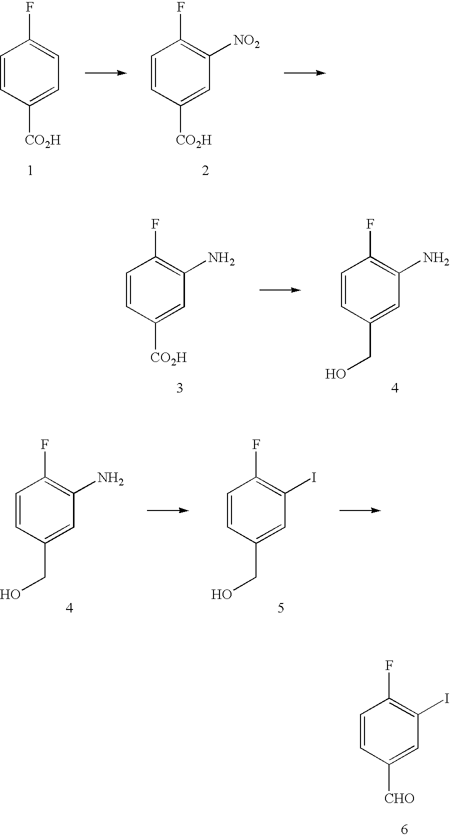

Another process for producing 4-fluoro-3-iodobenzaldehyde is shown in Scheme II. There are disadvantages to this process as 4-fluoro3-bromo-benzaldehyde is relatively expensive as a starting material and the process requires low temperature conditions.

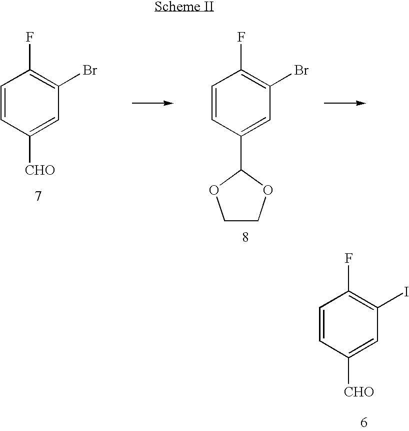

The present invention relates to a process for producing an intermediate that is used to make a dihydropyridine potassium channel opener.

### DETAILED DESCRIPTION OF THE INVENTION

The present invention relates to an efficient synthesis of 4fluoro-3-iodo-benzaldehyde by iodinating 4-fluoro-benzaldehyde. 4-Fluoro-3-iodo-benzaldehyde is a key intermediate in the manufacture of the potassium channel opener, 5-(4-fluoro-3-iodophenyl)-5,10 dihydro-1H,3H-dipyrano[3,4-b:4,3-e]pyridine-4,6(7H,9H) dione.

The present invention relates to an improved process for iodinating benzaldehydes. As shown in Scheme III, the improved process allows for a one-step procedure for synthesizing 4 fluoro-3-iodo-benzaldehyde. 4-Fluoro-3-iodobenzaldehyde is synthesized by combining 4-fluorobenzaldehyde and N-iodosuccinimide in an acid medium. Preferably, the 4Fluoro-3-iodobenzaldehyde and N-iodosuccinimide are in a 1:1.2 equivalents ratio respectively.

Suitable acids for use in the present invention include organic acids and inorganic acids. An example of organic acids suitable for use in the present invention includes, but is not intended to be limited to, trifluoromethanesulfonic acid.

Inorganic acids are suitable for use in the present invention as well. Inorganic acids suitable for use in the present invention include, but is not intended to be limited to, nitric acid, sulfuric acid, and hydrochloride acid.

The inorganic acid may be used in combination with acetic acid as well. Acetic acid is used for solubility purposes. A strong inorganic acid/acetic acid combination is suitable for use in the present invention. Preferably, the inorganic acid/acetic acid combination is in a 1:1 ratio.

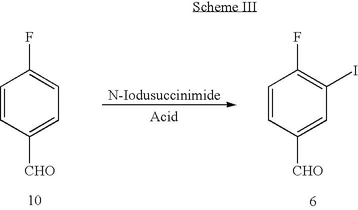

### EXAMPLE 1

4-Fluorobenzaldehyde (40.0 g), N-iodosuccinimide (87.1 g) and acetic acid (80 mL) were added to a flask. Sulfuric acid (80 mL) is added slowly, maintaining the temperature below 40° C. The resulting mixture was stirred for 2.5 h at 40° C. and then after cooling to 10° C., water (400 mL) was added, maintaining the temperature below 35° C. After stirring for 30 min at room temperature the slurry was filtered and the resulting solid washed with water (80 mL). The wetcake was dissolved in 380 g ethyl acetate/heptane (1:1 v/v containing 250 ppm BHT) and the solution was washed with 10% aqueous sodium thiosulfate (204 g), then with 10% aqueous sodium carbonate (214 g), followed by 200 g water.

The solution was concentrated to approximately 40 mL, and 148 g heptane (containing BHT) was added. This was distilled again to approximately 40 mL, to remove all of the ethyl acetate. Heptane (265 g) was added and the resulting mixture heated to 55° C. to dissolve the solids The solution was cooled to approximately 40° C. and seed crystals were added. Cooling was continued until the temperature reached 5° C. and then the slurry was filtered. After washing the wetcake with cold heptane, the product was dried in a vacuum oven at 40° C.

We claim:

1. A process for producing 4-fluoro-3-iodobenzaldehyde by combining 4-fluorobenzaldehyde, N-iodosuccinimide in an acid medium.

2. A process of claim 1 wherein said acid is an organic acid.

3. A process of claim 2 wherein said organic acid is trifluoromethanesulfonic acid.

4. A process of claim 1 wherein said acid is an inorganic acid.

5. A process of claim 4 wherein said inorganic acid is selected from the group consisting of nitric, sulfuric, and hydrochloric acid.

6. A process of claim 1 wherein said acid is an inorganic acid in combination with acetic acid.

7. A process of claim 4 herein said inorganic acid/acetic acid combination is in a 1:1 ratio.
